(19) United States
(12) Patent Application Publication
(10) Pub. No.: US 2025/0155571 A1
(43) Pub. Date: May 15, 2025

(54) APPARATUS, SYSTEM AND METHOD FOR ULTRASONIC IMAGING AND TREATMENT OF TISSUE MICROPATHOLOGY

(71) Applicant: CALLIOPE BIOPHYSICS, LLC, Orem, UT (US)

(72) Inventor: Timothy Edwin Doyle, Orem, UT (US)

(21) Appl. No.: 18/945,451

(22) Filed: Nov. 12, 2024

Related U.S. Application Data
(60) Provisional application No. 63/597,454, filed on Nov. 9, 2023.

(51) Int. Cl. G01S 15/89; A61B 8/08; A61B 17/32; G01V 1/02; G01V 1/18; G01V 1/52

(52) U.S. Cl. CPC G01S 15/8925; A61B 8/483; A61B 17/320068; G01S 15/8993; G01V 1/02; G01V 1/18; G01V 1/52; A61B 2017/320069

(57) ABSTRACT

An apparatus, system and method are disclosed for two-dimensional (2D) and three-dimensional (3D) imaging including ultrasonic imaging and treatment of tissue micropathology within a body. An apparatus may include two or more arrays of ultrasonic transducers with a volume of tissue or other region or object between the arrays. Groups of elements in the first array are pulsed together, with the pulses time-delayed (phased) to create ultrasonic or electric beam patterns that focus the beam energy through the tissue, region or object onto individual elements of the second array. Computed tomography (CT) techniques process the signals to reconstruct a 2D or 3D structure. Micropathology within a tissue may be treated by transmitting pulses from both arrays, by pulsing groups of elements together, and/or by phasing the pulses to focus on and ablate the region of micropathology.

### FIELD OF THE INVENTION

This invention relates primarily to surgical instrumentation and more particularly relates to ultrasonic diagnostic imaging and ultrasonic treatment of tissue micropathologies.

### BACKGROUND

The most common ultrasonic imaging method in medical diagnostics is phased array imaging. In conventional phased array imaging, different elements in an ultrasonic array are pulsed at different times (phased) to form, focus, and steer the ultrasound into a beam. This focal point is then scanned through the tissue in a raster fashion to construct an image of structures in the tissue that produce an acoustic impedance mismatch and therefore a reflection. If the structures are much larger than the wavelength of the interrogating ultrasound, such as the boundaries between organs or tumors with surrounding tissue, then a specular reflection is returned. If the structures are approximately the same size or smaller than the wavelength of the interrogating ultrasound, such as tissue microstructures, then a diffuse reflection is returned. Using breast tissue as an example, tissue microstructures may include ductules, lobules, fibrous tissue, microcalcifications, and cell clusters. At each focal point scanned in the tissue, pulse-echo reflections propagate back to the same array that transmitted the pulses, where they are received and processed to construct an image.

Image interpretation plays a principal role in phased array imaging for diagnosing anomalous structures in the human body. The analysis may include determining the morphology of the structure (e.g., smooth versus irregular boundaries of a breast tumor), the presence of internal structure (e.g., the presence of calcifications within a breast tumor), or the echogenicity of the structure (e.g., a fluid-filled cyst with no internal diffuse scattering versus a solid tumor with internal diffuse scattering). Another method for analyzing phased array data is Quantitative Ultrasound (QUS). This method looks at gross features of the ultrasonic spectrum, such as its slope, to determine parameters such as the size or the spacing of the ultrasonic scatterers in the probed tissue region.

An alternative to the phased array method in diagnostic imaging is computed tomography (CT). CT scans are most widely used with x-rays to produce 2D images that provide a series of cross-sections (slices) of the internal anatomy of a human body. These cross-sections can also be assembled to provide a 3D image of the internal anatomy of a human body. CT scans are also widely used in many other fields beyond medicine to image the internal structure of diverse objects, including solid manufactured objects and parts in nondestructive evaluation (NDE), fossils in paleontology, artifacts in archeology, drill cores in geology, and parcels and baggage in aviation security (for explosives and weapons).

CT imaging is a distinctly different process as compared to phased array imaging. Unlike phased array imaging, which is a reflection or pulse-echo approach, CT is a transmission approach. CT typically uses a single x-ray source, but in some cases, CT can also use multiple sources. The x-rays are typically detected by an x-ray detector (receiver) array on the opposite side of the object being imaged. The principal difference, however, between CT and phased array imaging is in how the rays or waves are focused. Since x-rays cannot be formed in beams, focused, or steered using phasing methods because of their ultrashort wavelengths, they are typically broadcast through the interrogated tissue in a uniform, spherical (diverging) manner, similar to a point light source. Focusing is achieved by collimating the transmitted x-rays at the x-ray detectors (receivers) using a lead plate with an array of straight channels in front of the detectors. Sometimes focusing is achieved by collimating the x-rays at the source (transmitter), again using a lead plate with an array of straight channels, but in this case, in front of the source.

To image the interior of an object with CT imaging, the source plus detector array configuration is rotated around and/or translated across the object to produce multiple x-ray projections in the object that can sample each 2D pixel or 3D voxel in the image area or volume with multiple projections at various orientations. The detector signals (measurements), the geometric relationships between the projections and the image pixels (voxels), and the final, unknown image values all comprise an enormous set of simultaneous equations, which are solved using a tomography algorithm. Various algorithms for reconstructing tomographic images include back projection methods, algebraic reconstruction techniques (ART), integral approaches, and iterative methods.

Ultrasonic imaging is typically not used in a CT mode of operation for medical imaging of the human body. Both the thickness of the human body and the difficulty of transmitting ultrasound through bones; dense tissue; multiple tissue layers; and air in lung and gastrointestinal organs renders it nearly impossible to produce a full-body CT scan with ultrasound. Any signals that managed to propagate through the body would be extremely weak and contaminated with artifacts from unwanted scattering and reflections from bones and air. An exception is the breast, which has relatively homogeneous tissue compared with the rest of the body (no bones or air). Such an experimental CT-scan device and method has been developed, constructed, and tested by other researchers for the localized imaging of female breasts immersed in a water bath.

However, both phased array and CT-scan approaches have limitations for optimal microimaging. A reflection based phased array does not transmit through tissue and inherent low signal amplitude limits CT-scan methods. Therefore, a need exists for microimaging tools and methods that combine the advantages and overcome the limitations of existing phased array and CT-scan approaches.

### SUMMARY

From the foregoing discussion, it should be apparent that a need exists for an apparatus, system, and method that improves CT-scan signal amplitude and also enhances the advantages of phased array scanning. Beneficially, such an apparatus, system, and method would enable real time evaluation and even treatment of micropathologies.

The present invention has been developed in response to the present state of the art, and in particular, in response to the problems and needs in the art that have not yet been fully solved by currently available microimaging tools and methods. Accordingly, the present invention has been developed to provide an apparatus, system, and method for 3D microimaging that overcome many or all of the above-discussed shortcomings in the art.

The merging of a phased array approach with a CT approach has distinct advantages over a solely phased array technique or a solely tomographic technique for imaging small tissue volumes (60-6000 mm³)* with high-frequency (HF) ultrasound (20-80 MHz). Phased array techniques are exclusively used for imaging in pulse-echo mode, where the ultrasonic reflection and back scattering properties of the 3D volume are imaged. In contrast, transmission tomography with a CT approach permits ultrasonic transmission properties to be measured and imaged, including ultrasonic wave speed, ultrasonic attenuation, viscoelastic properties, and microscopic structure from forward scattering spectral analysis.

*Note: 60 mm³ corresponds to a cubic volume with dimensions of about 4×4×4 mm, whereas 6000 mm³ corresponds to a cubic volume with dimensions of about 18×18×18 mm.

Ultrasonic transmission properties provide a more direct and mechanistic-based route for determining the micropathology of tissue with a high detection capability. For example, forward scattering spectral analysis with HF ultrasound has been found to be particularly sensitive to the diameters of nuclei in cells. An increase in nuclear size is a diagnostic characteristic of cancer, inflammation, and other pathologies. In one HF ultrasound study of 77 lymph nodes excised during breast conservation surgery, a multivariate analysis of two spectral analysis parameters, peak density and tortuosity, gave an 89.6% accuracy, 100% sensitivity, 88.4% specificity, and a p-value of 6.12×10⁻⁷ for the detection of metastatic breast cancer.

CT methods, however, have an inherent problem with low signal amplitude. This is due to the individual pairing of transmitting and receiving transducer elements in the ultrasonic arrays to produce a plurality of narrow pencil-beam ray patterns that fully cover the 3D tissue volume to be imaged. The technology provided herein solves this problem by pulsing groups of elements in the transmitting array together, phasing the pulses to constructively interfere to a focus at a single element in the receiving array, and then electronically scanning the focus of the beam to other elements in the receiving array. The phasing and resultant focusing of the beam patterns significantly amplifies the signal amplitudes at the receiving elements, while scanning the phased beam patterns across the receiving array produces a plurality of intersecting wide-beam patterns that can be processed with a CT algorithm to reconstruct the ultrasonic transmission properties of the 3D tissue volume.

Many common CT techniques, particularly those that use ionizing radiation such as x-rays, use a fan-beam geometry for the energy that penetrates and probes the region of interest. In comparison to these methods, the fan beam herein is inverted to focus energy onto the detector elements, and it is steered electronically. In x-ray CT methods, the penetrating energy is emitted from a single point source or transmitter. This energy diverges into a fan-shaped beam within an interrogated region and is received by an array of detector elements on the opposite side of the region. The beam is then steered by rotating and/or translating the source and detector array around the interrogated region.

In contrast, the apparatus provided herein emits the penetrating energy from an array of source elements. The energy is electronically formed into an inverted fan beam, or del beam (from the mathematical symbol V in vector calculus), by phasing (timing) the emissions from each of the source elements. The del beam propagates through the interrogated region and converges to a focal point on a single detector element on the opposite side of the region. The beam is then electronically steered onto other detector elements by modifying the phasing of the source elements.

As discussed above, in comparison to ultrasonic phased array methods, this apparatus provides information on the ultrasonic transmission properties of the interrogated region as opposed to its reflection properties. Additionally, whereas phased array methods image an interrogated region by scanning the ultrasonic focus across each point within the region's 3D volume, the apparatus herein images an interrogated region by scanning the ultrasonic focus across each detector element on a 2D array, and then computationally reconstructs a 3D image of the region from the overlapping projections of the del beams.

The apparatus disclosed herein significantly deviates from both conventional phased array ultrasound and conventional CT imaging. First, the apparatus of a conventional phased array system uses a single array of transducers to transmit, focus, and receive back ultrasonic waves propagated through the tissue. In contrast, the apparatus herein uses two or more separate arrays, with one array dedicated to transmitting and focusing the ultrasonic waves through the tissue, and with one or more other arrays dedicated to receiving the ultrasonic waves. Second, the system provided herein diverges from that of both phased array and CT imaging devices by simultaneously incorporating not only instruments to control the phases of the ultrasonic pulses emanating from the transmitting array, but also instruments to control the element sequencing, detection, processing, and tomographic image formation of ultrasonic signals from the receiving array. Finally, the method provided herein diverges from that of both phased array and CT imaging devices. Whereas conventional phased array devices use only the information reflected back from the focal point or focal plane of the ultrasonic waves, the technology herein uses the entire information collected by the ultrasonic waves as they propagate through the tissue. By treating the ultrasonic beams in the tissue as tomographic projections, the volume of the tissue can be imaged using tomographic reconstruction techniques.

Provided herein is an apparatus to interrogate the internal structure of a region or object, the apparatus comprising a transmitting array of discrete elements to transmit and focus electronic waves through an interrogated region or object, a receiving array for the electronic waves comprising at least one of a plurality of strip elements or a single large-area element, a software comprising a program to at least one of coordinate a phased pulsing of the transmitting elements, read the receiver elements, and topographically process the signals. In some embodiments the electronic waves are ultrasonic. In various embodiments the apparatus configures the ultrasonic waves as a triangular projection by phasing (timing) the emissions from each of the active discrete elements. The triangular projection may comprise an inverted fan beam, or del beam (∇).

Further provided herein is a software program for the apparatus. The software program sometimes comprises at least one of a projection module to generate electronic wave forms, a timing module to phase a plurality of ultrasonic wave transmissions, a configuring module to configure the ultrasonic wave as a triangular projection, an orientation module to create an inverted del beam, a detector module to receive and read a del beam focal point from a receiving array element, a steering module to electronically steer an inverted del beam focal point to a receiving array element, a focus beam scanning module to electronically scan the focus of the del beam to a plurality of elements in the receiving array, a wide beam pattern module to scan phased beam patterns across the receiving array to produce a plurality of intersecting wide-beam patterns, a storage module to collect and store signals from the receiving array elements, a processing module process the plurality of intersecting wide-beam patterns with a CT algorithm to reconstruct the ultrasonic transmission properties of the interrogated region or object, and a tomographic image module to create a 3D image of the interrogated region or object. Two or more modules of the software program are sometimes merged or combined. In some embodiments the software program further comprises an image fixing or projection module to record and/or distribute the image.

Also provided herein is a system to construct 2D and 3D ultrasonic images in an attenuating media, where the ultrasonic measurements and data are multifrequency and/or broadband in nature, and the 2D/3D ultrasonic images retain the multifrequency and/or broadband characteristics of the ultrasonic measurements and/or data, and a receiving array for the electronic waves comprising at least one of a plurality of strip elements or a single large-area element. In some embodiments the system comprises a tunable ultrasonic pulsing module, a controller that tunes the ultrasonic pulsing module for each frequency step in the multifrequency and/or broadband ultrasonic spectrum, a multiplexer that switches through the sensors in a transmitting sensor array and selects combinations of transmitting array elements to generate each ultrasonic beam geometry, a first ultrasonic sensor array that generates and broadcasts ultrasonic waves with multifrequency and/or broadband characteristics, a second ultrasonic sensor array that receives and detects ultrasonic waves with multifrequency and/or broadband characteristics, a multiplexer that switches through the sensors in the receiving sensor array and selects combinations of receiving array elements to correspond to each ultrasonic beam geometry, an ultrasonic receiving module to amplify, filter, and/or rectify the received ultrasonic signal from the selected receiving array elements, a computational device that calculates the frequency steps for a multifrequency and/or broadband ultrasonic spectrum, beam geometry combinations required to reconstruct a 2D or 3D image of the internal structure or property distribution of a medium, sensor element combinations for each ultrasonic beam geometry, and corresponding sensor element delays for each frequency step and for each beam geometry, a data storage device that stores the frequency steps, beam geometries, sensor element combinations, and sensor element delays, and a computational device that uses the beam pattern geometries, receiving element measurements, and broadband ultrasonic spectrum analysis to construct a 2D or 3D image of the internal structure or property distribution of the medium. In various embodiments system herein further comprises an image fixing and/or projection device to capture and/or distribute the constructed image.

Additionally provided herein is a method to increase the signal strength from a first transmitting sensor array, broadcasting ultrasound through an attenuating medium, to a second receiving sensor array, and obtaining two-dimensional and three-dimensional images of the internal structure or distribution of material, physical, chemical, or biological properties of the medium. In some embodiments the method comprises calculating a frequency for a broadband spectrum, selecting a frequency step for executing a measurement, tuning a pulser for the selected frequency step, selecting an ensemble of transmitting elements and receiving elements for a selected beam pattern, calculating delay times for pulsing the transmitting elements in the ensemble for the selected frequency, broadcasting ultrasound, pulsing the transmitting elements in the ensemble using a multiplexer, collecting and storing the signals from the receiving elements in the ensemble using a multiplexer, looping to stored frequencies to select the next frequency step, and using beam pattern geometries and receiver element measurements to reconstruct broadband ultrasonic CT images the broadband ultrasonic CT images are 2D, 3D or 4D.

In various embodiments of the method herein the medium is animal tissue, human tissue, or other tissue. The method sometimes further comprises the use of broadband spectrum analysis to construct a 3D pathology image of a tested tissue or region. The attenuating medium may comprise at least one of biological, mineral, geologic, rock, soil, landform, machine, construction, solid manufactured objects and parts in a nondestructive evaluation (NDE), and parcels and baggage in aviation security (for explosives and weapons) or other attenuating medium.

Broadband spectrum analysis may be used construct a 3D inclusion image of the medium. An inclusion sometimes comprises at least one of oil, precious metal, fossil, drill core, industrial metal, precious stones, water, or other inclusion. The method here may comprising use of broadband spectrum analysis to interrogate a geological structure or feature. The geological structure or feature may comprise at least one of a fault line, a sink hole, a cavern, a well, a rock type, a soil type, a geological stratum, an archaeological structure, or other geological structure or feature.

Also provided herein is a method to construct 2D and 3D ultrasonic images in attenuating media, where the ultrasonic measurements and data are multifrequency and/or broadband in nature, and the 2D/3D ultrasonic images retain the multifrequency and/or broadband characteristics of the ultrasonic measurements and/or data. In various embodiments the method provided comprises, providing a first transmitting sensor array, broadcasting ultrasound through an attenuating medium to a second receiving sensor array, providing a first ultrasonic sensor array that generates and broadcasts ultrasonic waves with multifrequency and/or broadband characteristics, providing a second ultrasonic sensor array that receives and detects ultrasonic waves with multifrequency and/or broadband characteristics, and obtaining two-dimensional and three-dimensional images of an internal structure or distribution of material, physical, chemical, or biological properties of the medium. The internal structure of the biological properties of the medium sometimes comprises a pathology.

Various embodiments of the technology disclosed herein provide the capability to treat pathological tissue at the microscopic level by using phased array operation of the transmitting array to focus on a micropathological region in the tissue and to ablate the region with the focused ultrasound intensity. In this therapeutic mode, the receiving array may also be used as a second transmitting array to focus on the same micropathological region and contribute to the ablation of the region with the focused ultrasound intensity.

In certain embodiments the disclosed technology may be applied to the ultrasonic imaging of the internal structure and ultrasonic transmission properties of animal tissue, plant tissue, nonliving manufactured materials (including but not limited to metals, ceramics, glasses, plastics, elastomers, and composites), and nonliving natural materials (including but not limited to rocks, soils, drill cores, and fossils). At lower acoustic frequencies in the audible and infrasonic range, the technology herein may be applied to oceanographic imaging with floating and submerged acoustic arrays, and geologic imaging of the Earth's upper crust using cross-well borehole methods.

An embodiment of the technology herein may also be extended to electromagnetic (EM) imaging using frequency bands with wavelengths amenable to phased array focusing, including radio waves (atmospheric and geologic imaging), microwaves (NDE of nonliving manufactured materials), and terahertz or far infrared (FIR) waves (micropathology imaging). Combining HF ultrasonic elements with FIR elements in both the transmitting and receiving arrays may provide increased imaging, diagnostic, and therapeutic capabilities for mapping and treating the micropathology of a tissue volume.

Reference throughout this specification to features, advantages, or similar language does not imply that all of the features and advantages that may be realized with the present invention should be or are in any single embodiment of the invention. Rather, language referring to the features and advantages is understood to mean that a specific feature, advantage, or characteristic described in connection with an embodiment is included in at least one embodiment of the present invention. Thus, discussion of the features and advantages, and similar language, throughout this specification may, but does not necessarily, refer to the same embodiment.

Furthermore, the described features, advantages, and characteristics of the invention may be combined in any suitable manner in one or more embodiments. One skilled in the relevant art will recognize that the invention may be practiced without one or more of the specific features or advantages of a particular embodiment. In other instances, additional features and advantages may be recognized in certain embodiments that may not be present in all embodiments of the invention.

These features and advantages of the present invention will become more fully apparent from the following description and appended claims or may be learned by the practice of the invention as set forth hereinafter.

### BRIEF DESCRIPTION OF THE DRAWINGS

In order that the advantages of the apparatus, system, and method herein will be readily understood, a more particular description of the technology briefly described above will be rendered by reference to specific embodiments that are illustrated in the appended drawings. Understanding that these drawings depict only typical embodiments of the technology and are not therefore to be considered to be limiting of its scope, the invention will be described and explained with additional specificity and detail through the use of the accompanying drawings, in which:

FIG. 1A, FIG. 1B, and FIG. 1C are line drawings illustrating an embodiment of the principle of phased array focusing of ultrasonic waves onto an object, with the use of a sequence of three timeframes and using an overlay of all three timeframes in accordance with the present invention; sequence of three time-frames in accordance with the present invention;

FIG. 1D is a line drawing illustrating an embodiment of an overlay of all three timeframes wherein the constructive interference of three wavefronts amplifies and focuses the ultrasonic intensity onto the object in accordance with the present invention;

FIGS. 2A, 2B, and 2C are line drawings depicting an embodiment of the principle of computed tomography (CT) x-ray imaging of the interior of an object and depicting a single, transmitting x-ray source, emitting x-rays in a fan beam pattern to an arc-shaped array of receiving detectors a sequence of three timeframes wherein rotation of the source and receiver array configuration produces multiple projections (x-ray paths) through the object at multiple angles in accordance with the present invention;

FIG. 3A, FIG. 3B, and FIG. 3C are line drawings illustrating an embodiment of a phased array focusing of ultrasonic waves from a transmitting array onto a single element of an oppositely facing receiving array, with the use of a sequence of three timeframes and with an overlay of all three timeframes in accordance with the present invention;

FIG. 3D is a line drawing depicting an embodiment of the constructive interference of three wavefronts that amplifies and focuses the ultrasonic intensity onto the receiving array element in accordance with the present invention;

FIGS. 4A and 4C are line drawings depicting an embodiment of the ray paths of the ultrasonic waves emanating from two or more elements of the transmitting array and focused onto a single receiving array in accordance with the present invention;

FIGS. 4B and 4D are line drawings depicting an embodiment of the ensemble of ray paths for each transmitter array/receiving element configuration that produces a del beam projection through the region's interior in accordance with the present invention;

FIG. 4E is a line drawing depicting an embodiment of a collection of two or more del beam projections through the region's interior that provides data for the tomographic reconstruction of an image of the region's interior structure or properties in accordance with the present invention;

FIG. 5A is a line drawing depicting and embodiment of a 2D transmitting array and a 2D receiving array in accordance with the present invention;

FIG. 5B, FIG. 5C and FIG. 5D are line drawing depicting an embodiment of various combinations and orientations of del beam projections to a single element in the receiving array in accordance with the present invention;

FIG. 6A and FIG. 6B are line drawing depicting an embodiment of combinations and orientations of del beam projections from a 2D transmitting array to a single element in a 2D receiving array in accordance with the present invention;

FIG. 7 is a high-level schematic flowchart for an embodiment of the phased-emission computed-tomography (PECT) imaging method in accordance with the present invention;

FIG. 8 is a schematic flowchart for an embodiment of the PECT imaging system in accordance with the present invention;

FIG. 9 is a schematic flowchart for an embodiment of a method for the PECT imaging system that includes additional elements for broadband spectral operation in accordance with the present invention;

FIG. 10A is a line drawing depicting a horizontal cross-section through a human head, with the skull and brain structure outlined. On opposite sides of the head are depicted embodiments of two conformable ultrasonic transducer arrays. The conformable array depicted the left side is an embodiment of the transmitting array, and the conformable array depicted on the right side is an embodiment of the receiving array in accordance with the present invention;

FIG. 10B, is a line drawing depicting an embodiment of two del beam projections emanating from the transmitting array and focused on two different transducer elements of the receiving array, in accordance with the present invention;

FIG. 11A is a line drawing depicting embodiments of a 2D transmitting array and of a 2D receiving array consisting of resistive electrode line transducers for imaging the structure or properties of the 3D region existing between the two arrays;

FIG. 11B, FIG. 11C, and FIG. 11D are line drawings depicting embodiments of various combinations and orientations of del beam projections to a single line transducer in the receiving array in accordance with the present invention;

FIG. 12A is a line drawing depicting an embodiment of a 2D transmitting array and a single receiving element comprising a resistive electrode large-area transducer for imaging the structure or properties of the 3D region existing between the two arrays in accordance with the present invention;

FIG. 12A, FIG. 12B, FIG. 12C and FIG. 12D are line drawings depicting embodiments of various combinations and orientations of del beam projections to the single receiving transducer in accordance with the present invention;

FIG. 13A is a line drawing of a cross-section of a resistive electrode line transducer, depicting an embodiment of the structure of the transducer, including a focus point of a del beam on the transducer, and the direction of the electrical current flowing from the region of the piezoelectric transducer where the del beam has created a localized charge separation according to the present invention;

FIG. 13B is a line drawing depicting a close-up of an embodiment of the resistive electrode line transducer at the focus point of the del beam, and displays the localized charge separation on isolated conductive electrodes embedded in the resistive electrode material, in accordance with the present invention;

FIG. 14 is a software diagram flow chart depicting an embodiment of a software to interrogate the internal structure of a region or object in accordance with the present invention;

FIG. 15 is a method diagram of an embodiment of a method for obtaining two-dimensional and three-dimensional images of the internal structure or distribution of material, physical, chemical, or biological properties of a medium in accordance with the present invention.

FIG. 16 is a flow chart depicting an embodiment of a method to increase the signal strength from a first transmitting sensor array by broadcasting ultrasound through an attenuating medium, to a second receiving sensor array, and obtaining two-dimensional and three-dimensional images of the internal structure or distribution of material in the attenuating medium in accordance with the present invention.

FIG. 17 is a line diagram depicting an embodiment of an apparatus in accordance with the present invention.

### DETAILED DESCRIPTION

Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

Furthermore, the described features, structures, or characteristics of the invention may be combined in any suitable manner in one or more embodiments. In the following description, numerous specific details are provided, such as examples of programming, software modules, user selections, network transactions, database queries, database structures, hardware modules or devices etc. to provide a thorough understanding of embodiments of the invention. One skilled in the relevant art will recognize, however, that the invention may be practiced without one or more of the specific details, or with other methods, components, materials, and so forth. In other instances, well-known structures, materials, or operations are not shown or described in detail to avoid obscuring aspects of the invention.

The schematic flow chart diagrams included herein are generally set forth as logical flow chart diagrams. As such, the depicted order and labeled steps are indicative of one embodiment of the presented method. Other steps and methods may be conceived that are equivalent in function, logic, or effect to one or more steps, or portions thereof, of the illustrated method. Additionally, the format and symbols employed are provided to explain the logical steps of the method and are understood not to limit the scope of the method. Although various arrow types and line types may be employed in the flow chart diagrams, they are understood not to limit the scope of the corresponding method. Indeed, some arrows or other connectors may be used to indicate only the logical flow of the method. For instance, an arrow may indicate a waiting or monitoring period of unspecified duration between enumerated steps of the depicted method. Additionally, the order in which a particular method occurs may or may not strictly adhere to the order of the corresponding steps shown.

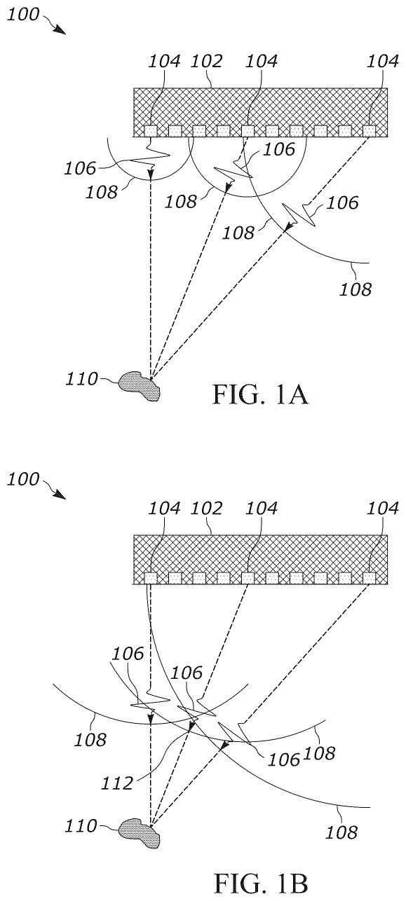

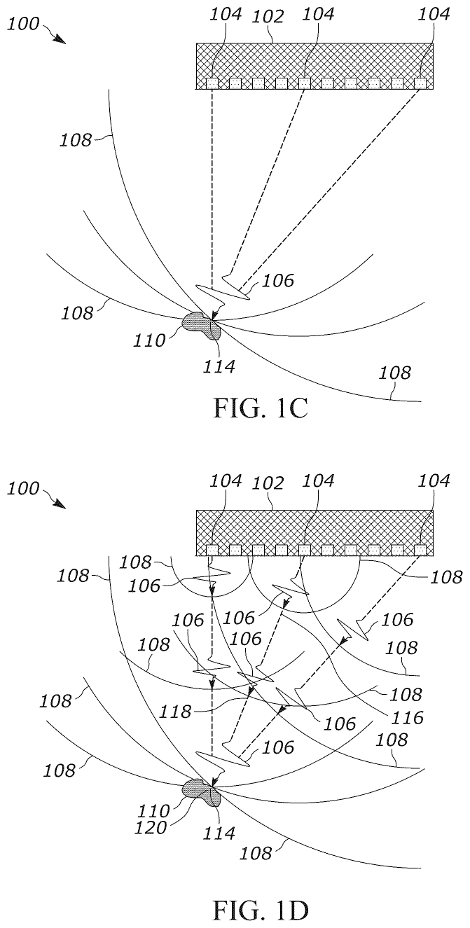

In conventional phased array imaging, different elements in an ultrasonic array are pulsed at different times (phasing) to form, focus, and steer the ultrasound into a beam. FIG. 1A depicts an embodiment of a linear, one-dimensional (1D), ultrasonic array 102 for creating a 2D image of the ultrasonic reflection properties within a tissue volume.

FIG. 1A depicts an embodiment of the pulsing of three different array elements 104 with three different time-delays or phases creating pulses 106. The semicircles 108 in the figure depict the spherical wavefronts of the pulses 106 as they spread into the tissue 110.

FIG. 1B depicts an embodiment of the pulses 106 beginning to converge 112 as they propagate through the tissue 110. FIG. 1C depicts the eventual focus of the pulses 106 to a point 114. In various embodiments this point 114 is then scanned through the tissue 110 in a raster fashion to construct an image of structures within the tissue 110 that produce an acoustic impedance mismatch and therefore a reflection.

FIG. 1D depicts an embodiment of the wave propagation of the pulses 106 at all three different times in phased array operation: first, immediately following pulsing 116, second, converging of the pulses 118, and third focusing 120 of the pulses to a focal point 114. At each focal point 114 scanned in the tissue 110, pulse-echo reflections may propagate back to the same array 104 that transmitted the pulses 106, where they may be received and processed to construct the image.

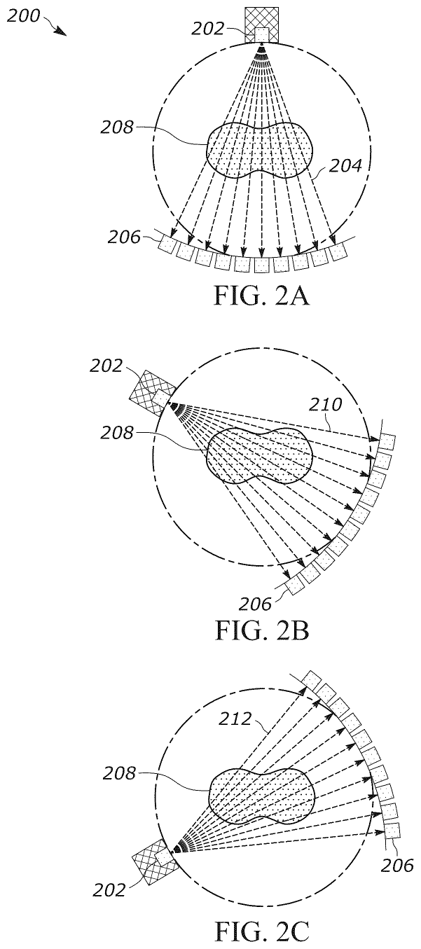

FIGS. 2A, 2B, and 2C depict an embodiment of the phased-emission stage of operation of an embodiment of PECT for 2D imaging in accordance with the present invention. FIG. 2A is a line drawing depicting a single, transmitting x-ray source 202, emitting x-rays in a fan beam pattern 204 that pass through a region or object 208 to be interrogated, and then intercepted by an arc-shaped array of receiving detectors 206 on the opposite side of the region or object 208. FIG. 2B and FIG. 2C depict embodiments of rotation of the source 202 and receiver array configuration 206, producing multiple projections (x-ray paths) through the region or object 208 at multiple angles, 210 and 212, providing data for the tomographic reconstruction of an image of the object 208's interior structure or properties such as x-ray density in accordance with the present invention.

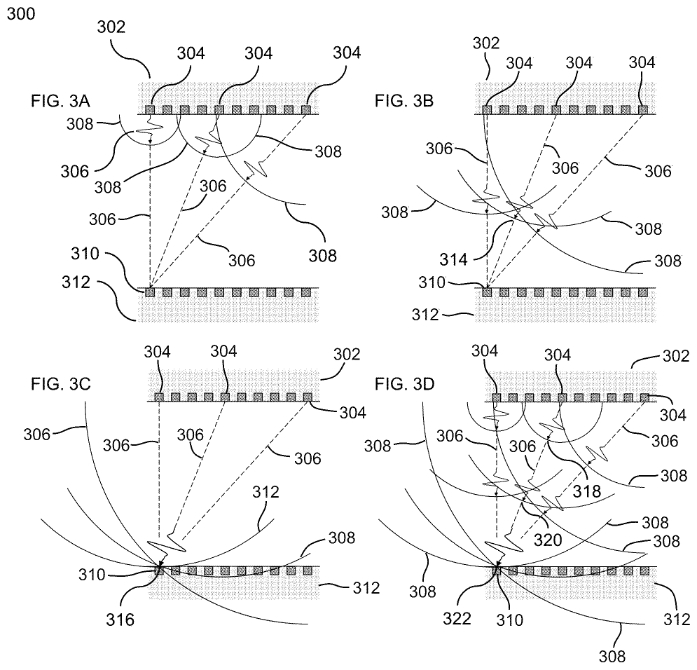

FIGS. 3A, 3B, and 3C are line drawings depicting embodiments of the phased-emission stage of operation of an embodiment of PECT for 2D imaging in accordance with the present invention.

FIG. 3A depicts a linear, 1D, ultrasonic transmitting array 302 and a linear, 1D, ultrasonic receiving array 312 for creating a 2D image of the ultrasonic transmission properties within a region or object to be interrogated 208, in some embodiments tissue. FIG. 3A depicts embodiments of three different transmitting array elements 304 which are pulsed with three different time-delays or phases creating pulses 306. The semicircles 308 in the figure depict the spherical wavefronts of the pulses as they spread into the region or object to be interrogated 208.

FIG. 3B depicts an embodiment of the pulses 306 as they propagate through the region or object to be interrogated 208 and begin to converge 314. FIG. 3C depicts an embodiment of the eventual focus to a point 316 on a single detector element 310 in the ultrasonic receiving array 312. FIG. 3D depicts an embodiment of the wave propagation of the pulses 306 at all three different times in the phased-emission stage of the PECT operation: First, immediately following pulsing 318; second, converging 320 of the pulses 306; and third focusing of the pulses 306 to a point 322 on the receiving array element 310.

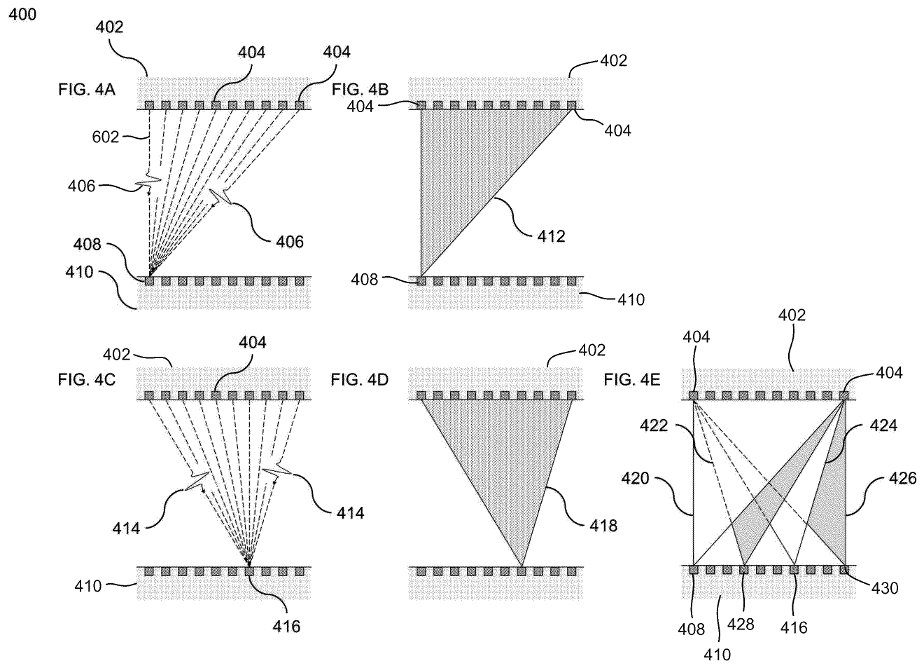

FIGS. 4A, 4B, 4C, 4D, and 4E are line drawings depicting embodiments of both the phased-emission stage and computed-tomography stage of an embodiment of PECT for 2D imaging in accordance with the present invention.

FIG. 4A depicts an embodiment of a linear, 1D, ultrasonic transmitting array 402 and a linear, 1D, ultrasonic receiving array 410 for creating a 2D image of the ultrasonic transmission properties within a region or object to be interrogated 208, including a volume of tissue. In certain embodiments phased emissions from the ten transmitting array elements 404 generate pulses 306 with ray paths 406 that converge upon a single receiving array element 408. FIG. 4B depicts an embodiment of the formation by the ensemble of converging ray paths of an inverted fan-beam or del-beam projection 412 in an interrogated region or object 208.

FIG. 4C and FIG. 4D depict embodiments of the creation of a second del-beam projection 412 in the interrogated region or object. Phased emissions from the ten transmitting array elements 404 may generate pulses 306 with ray paths 414 that differ from those of the first projection 406, and that may converge upon a different receiving array element 416 than that of the first projection 408. The ensemble of different ray paths 414 may form a second del-beam projection 418 with a different geometry and coverage than that of the first projection 412.

FIG. 4E depicts an embodiment of an ensemble of different projections 420, 422, 424, 426 in the interrogated region or object 208, created by sequentially scanning the focal point of the phased emissions across the array elements 404 of the ultrasonic receiving array 410. In various embodiments, with a sufficient number of overlapping projections and the use of CT algorithms, a 2D image may be reconstructed from the projection geometries and the ultrasonic measurements read from each array element. Such CT algorithms include but are not limited to back projection methods, algebraic reconstruction techniques (ART), integral approaches, and iterative methods.

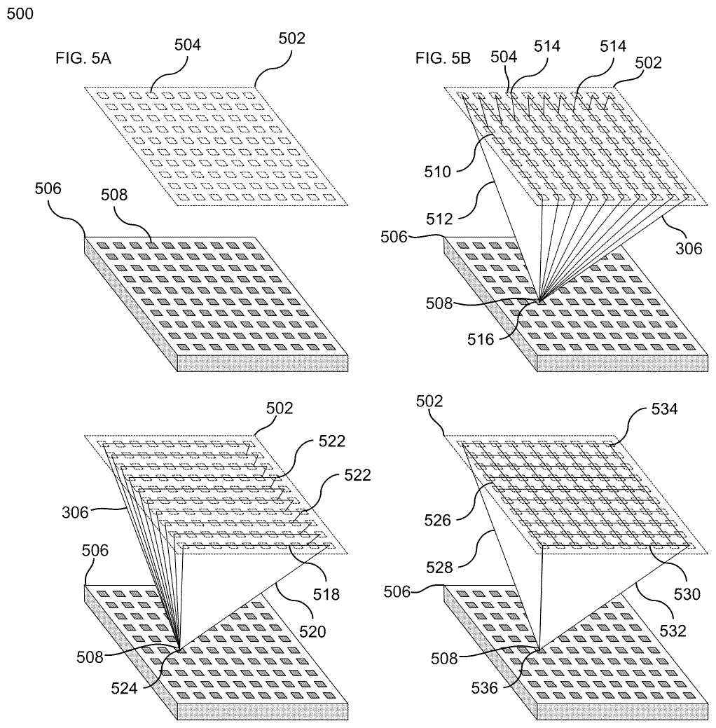

FIGS. 5A, 5B, 5C, and 5D are line drawings depicting embodiments of the phased-emission stage of operation of PECT for 3D imaging in accordance with the present invention.

FIG. 5A depicts a 2D, square-grid, ultrasonic transmitting array 502 with transmitting elements 504 and a 2D, square-grid, ultrasonic receiving array 506 with transmitting elements 508 for creating a 3D image of the ultrasonic transmission properties within an interrogated region or object 208.

FIG. 5B depicts an embodiment in which the transmitting array elements 504 are pulsed in groups 514, each group 510 comprising 10 elements and aligned as a row in the y-direction of the square-grid array. In certain embodiments the 10 elements 504 within each group 510 are pulsed with different time-delays or phases to focus the pulsed ultrasonic emissions 306. As the pulses 306 propagate through the interrogated region or object 208, they may converge to a point 516 onto a single detector element 508 in the ultrasonic receiving array 506, thus forming an inverted fan-beam, or del-beam projection 412 in the interrogated region or object 208.

In various embodiments projection 512 generates a measurement value at the receiving element 508. Sequential pulsing and phasing of transmitting elements 504 in other groups 514 create additional projections. These multiple projections may be combined into an ensemble of tomographic measurements. Refocusing the del projections 412 onto other receiving elements 508 of the receiving array 506 may expand the ensemble of measurements to cover the full volume of the interrogated region or object 208, thereby enabling full 3D tomographic reconstruction of the interior of the region or object 208.

With the use of a range of phasing combinations, both on-axis and off-axis focusing of the ultrasonic pulses may be achieved, resulting in 2D projections 514 that may be either perpendicular or non-perpendicular to the 2D, square-grid, transmitting array 502.

FIG. 5C depicts an embodiment wherein the transmitting array elements 504 are again pulsed in groups 522, each group 518 consisting of 10 elements, but in certain embodiments aligned as a row in the x-direction of the square-grid array. The 10 elements 504 within each group 518 may be pulsed with different time-delays or phases to focus the ultrasonic emissions. As the pulses 306 propagate through the interrogated object or region 208, the projection 520, 532, converges to a point 524, 536 onto a single detector element 508, in the ultrasonic receiving array 506, thus forming an inverted fan-beam, or del-beam, projection 412 in the interrogated region or object 208.

FIG. 5D depicts certain embodiments of the combining of projections 520, 512, generated from pulsing transducers 504 in rows aligned in both the y-direction 526 and x-direction 530, and the respective projections 528 and 532 that are created. Refocusing the focal point 536 of all of the projections onto other receiver elements 508 of the receiving array 506 may produce an ensemble of projections 528₊ₙ, 536₊ₙ, that fill the volume residing between the transmitting array 502 and receiving array 506. This enables the use of 3D tomographic reconstruction techniques to image the interior of the interrogated region or object 208.

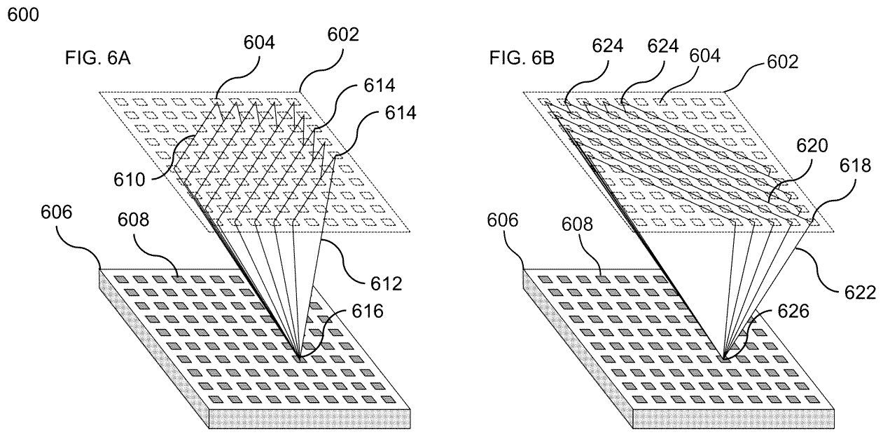

FIGS. 6A and 6B are line drawings depicting an embodiment of the phased-emission stage of operation of an embodiment of PECT for 3D imaging in accordance with the present invention. FIG. 6A depicts an embodiment of a 2D, square-grid, ultrasonic transmitting array 602 and a 2D, square-grid, ultrasonic receiving array 606 for creating a 3D image of the ultrasonic transmission properties within a tissue volume. In the embodiment FIG. 6A, the transmitting array elements 604 are pulsed in groups 614, each group 610 consisting of a variable number of elements and aligned as a row in a diagonal direction of the square-grid array. The elements 604 within each group 610 may be pulsed with different time-delays or phases to focus the pulsed ultrasonic emissions 306. As the pulses 306 propagate through the interrogated region or object 208 they may converge to a point 616 onto a single detector element 608 in the ultrasonic receiving array 606, thus forming an inverted fan-beam, or del-beam, projection 412 in the interrogated region or object 208.

Each projection 612 may generate a measurement value at the receiving element 608. Sequential pulsing and phasing of transmitting elements in other groups 614 may create additional projections 612. In some embodiments these multiple projections 612 are combined into an ensemble of tomographic measurements. Refocusing the del projections 412 onto other receiving elements 608 of the receiving array 606 may expand the ensemble of measurements to cover the full volume of the interrogated region or object 208, thereby enabling full 3D tomographic reconstruction of the interior of the region or object 208.

With the use of a range of phasing combinations, both on-axis and off-axis focusing of the ultrasonic pulses may be achieved, resulting in 2D projections 614 that may be either perpendicular or non-perpendicular to the 2D, square-grid, transmitting array 602.

FIG. 6B depicts an embodiment wherein the transmitting array elements 604 are again pulsed in groups 624, each group 620 comprising a variable number of elements, but in certain embodiments aligned as a row perpendicular to the diagonal direction of the rows shown in FIG. 6A. The elements 618 within each group 620 may be pulsed with different time-delays or phases to focus the pulsed ultrasonic emissions 306. As the pulses 306 propagate through the interrogated region or object 208, the projection 612, 622 may converge to a point 626 onto a single detector element 608 in the ultrasonic receiving array 606, thus forming an inverted fan-beam, or del-beam, projection 412 in the interrogated region or object 208.

In various embodiments combining the projections, 612 and 622, generated from pulsing transducers 604 in rows aligned in two different diagonal directions, 614 and 624, creates a plurality of projections 612, 622 from which images may be reconstructed with tomographic techniques. Refocusing the focal point, 616 and 626, of all of the projections 612₊ₙ, 622₊ₙ onto other receiver elements 608 of the receiving array 606 may produce an ensemble of projections that fill the volume residing between the transmitting array 602 and receiving array 606. This enables the use of 3D tomographic reconstruction techniques to image the interior of the interrogated region or object.

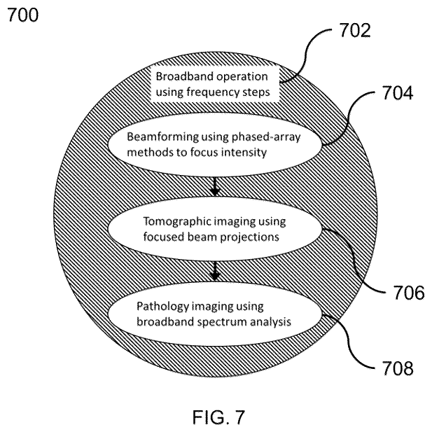

FIG. 7 depicts an embodiment of a high-level schematic flowchart for an embodiment of the phased-emission/computed-tomography (PECT) method for imaging tissue micropathology in accordance with the present invention. Propagation of ultrasound through biological tissue produces a strong correlation between the frequency dependence of the ultrasonic signal (also known as the signal's spectrum) and many aspects of the tissue's micropathology. These aspects may be closely linked to many disease processes in tissue, such as cancer or inflammation, and include the diameters of cell nuclei, the cell stiffness, the cell density, the cell viscoelasticity, and the material properties of the extracellular matrix.

In various embodiments Broadband operation of the PECT method 702 entails collecting broadband ultrasonic data in the frequency domain by advancing through discrete frequency steps. In certain embodiments, ultrasonic pulses are first generated with a frequency at a starting value F₀, and the pulses are phased to form a plurality of focused del-beam projections 704 that fill the interrogated region of the tissue. Second, the ultrasonic signals resulting from the del-beams propagating through the tissue are detected at the receiver array and processed into an F₀ image 706 using tomographic reconstruction techniques. The frequency of the ultrasonic pulses is sometimes then incrementally stepped to a new frequency F₁, a new ensemble of focused del-beam projections is generated 704 at the incremented frequency F₁, and the projection signals may be detected at the receiver array 706 and processed to create an F₁ image. This loop process may continue until N+1 3D tomographic images are generated for F₀, F₁, F₂, . . . , FN frequency steps. This series of frequency-dependent 3D images may then be juxtaposed and analyzed 708 with four-dimensional (4D) image processing tools (three spatial dimensions+one frequency dimension), producing a 3D map of the micropathology of the tissue in the interrogated region.

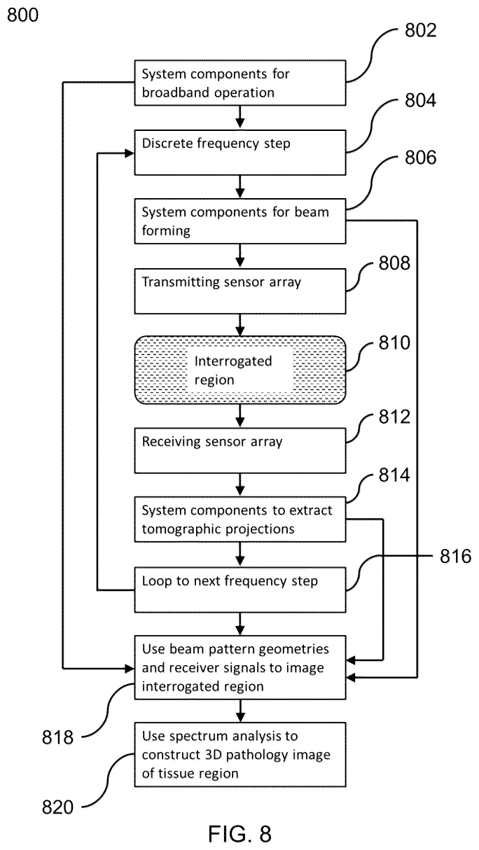

FIG. 8 depicts a medium-level schematic flowchart for an embodiment of the phased-emission/computed-tomography (PECT) system-based method for imaging tissue micropathology, specifically the system components for broadband operation 802. A discrete frequency is first chosen by a frequency selector 804 as the starting frequency for the data acquisition process. Next, system components 806 produce electrical pulses at the selected frequency, and phase-delay the pulses to generate del beams in the interrogated region. These electrical pulses are then communicated to the transmitting sensor array 808. The transmitting sensor array 808 converts the electrical pulses into ultrasonic pulses, which propagate through the interrogated region 810 as del beams and are collected by the receiving sensor array 812. In various embodiments the receiving sensor array 812 converts the ultrasonic pulses back into electrical signals. These electrical signals are then processed by system components 814 that extract the tomographic projection signals.

In some embodiments the data acquisition process then loops back 816 to the frequency selector 804, which increments the frequency to the next frequency step. The data acquisition process then proceeds to collect ultrasonic data through components 806 to 816 until data is acquired for all desired frequency steps. Once signals have been acquired for all desired projections and frequency steps, the signals may transfer to system component 818, which may use the beam pattern geometries and receiver signals to image the interrogated region in four dimensions (4D): three spatial dimensions and one frequency dimension. The 4D image data are sometimes then transferred to component 820, which may perform frequency spectrum analysis on the 4D images to construct 3D pathology images of the interrogated tissue region.

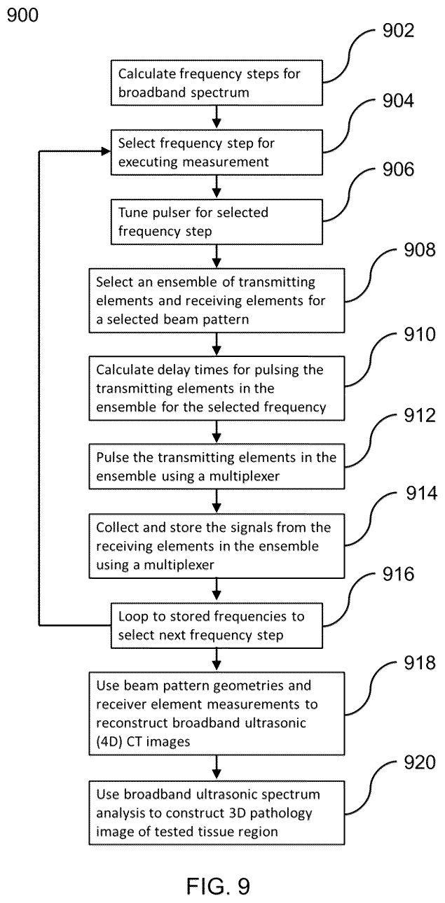

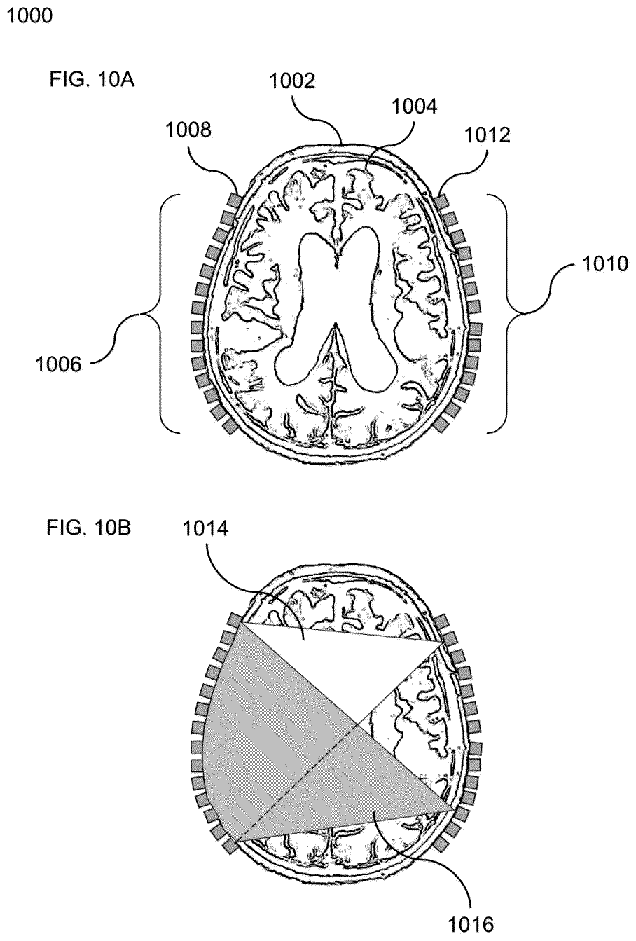

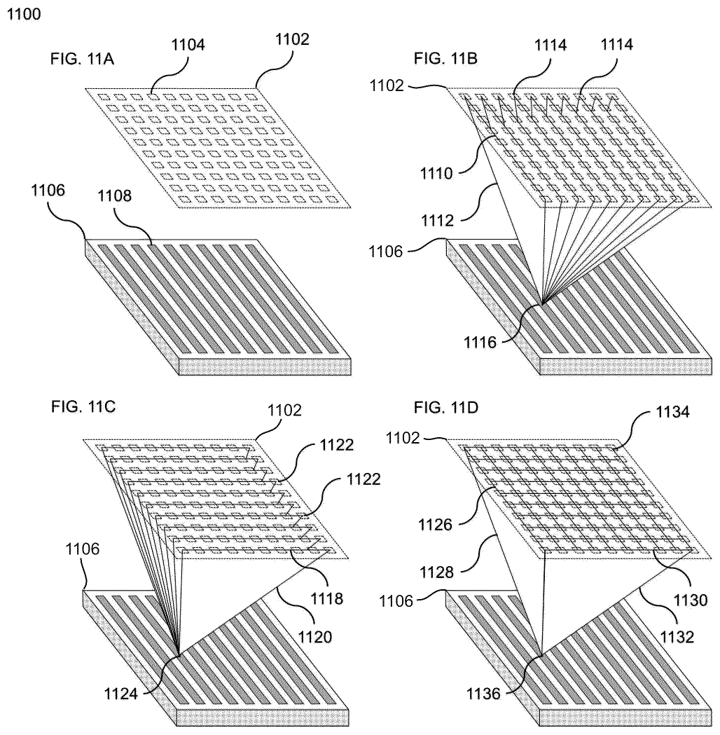

FIG. 9 depicts a low-level schematic flowchart for an embodiment of the phased-emission/computed-tomography (PECT) method for imaging tissue micropathology, specifically a method for broadband operation. In the depicted embodiment the start frequency and frequency steps for the data acquisition process are first calculated 902. The frequency step for executing the measurement is then selected 904. The pulser may be tuned to the selected frequency 906, and an ensemble of transmitting and receiving elements may be selected to form a specified beam pattern 908. Delay times may then be calculated for pulsing the transmitting elements in the ensemble for the selected frequency 910.

The transmitting elements in the ensemble may then be pulsed using a multiplexer, switch system, or other device 912 that channels the time-delayed pulses to the correct transmitting elements. The pulses generated by the ensemble of transmitting elements may produce ultrasonic waves in the interrogated region that focus onto receiving elements in the form of del-beam projections. In some embodiments the signals from the receiving elements in the ensemble are collected and stored using a multiplexer, switch system, or other device 914 that channels the ultrasonic signals from the correct receiving elements to a signal processing and storage device.

In certain embodiments the data acquisition process then loops back 916 to the frequency selector 904, which increments the frequency to the next frequency step. The data acquisition process may then proceed to collect ultrasonic data through steps 906 to 916 until data is acquired for all desired frequency steps. In various embodiments once signals have been acquired for all desired projections and frequency steps, the signals transfer to step 918, which uses the beam pattern geometries and receiver signals to image the interrogated region in 4D using computed tomography (CT) reconstruction methods. The 4D CT image data may then be transferred to step 920, which performs broadband frequency spectrum analysis on the 4D images to construct 3D pathology images of the interrogated tissue region.

FIGS. 10A and 10B are line drawings depicting the phased-emission stage of operation of an embodiment of PECT for noninvasively imaging the human brain ex vivo (from outside the human body) in accordance with the present invention.

FIG. 10A is a line drawing of a horizontal cross-section through a human head, with the skull 1002 and brain structure 1004 outlined. Depicted on opposite sides of the head are embodiments of two conformable ultrasonic transducer arrays, the transmitting array 1006 and the receiving array 1010. The transmitting array 1006 comprises individual ultrasonic transducers 1008 that are mounted on a flexible support frame that can conform around the curves of the head. Similarly, the receiving array 1010 comprises individual ultrasonic transducers 1012 that are mounted on a flexible support frame that can conform around the curves of the head. In various embodiments a position sensor is mounted on each transmitting transducer 1008 and receiving transducer 1012. Accurate knowledge of the transducers' locations may be essential for correctly calculating the phase delays to generate del beams within the skull and brain.

FIG. 10B depicts an embodiment of two del beam projections 1014 and 1016 emanating from the transmitting array 1006 and focused on two different transducer elements of the receiving array 1010, in accordance with the present invention. With knowledge of the transmitting transducer 1008 positions and receiving transducer 1012 positions, pulse delays may be calculated to produce del beams that focus onto the receiver transducers. Additional del beams focusing on other receiver elements may provide more information for tomographic reconstruction to more completely image the micropathology of the human brain.

The illustration depicts a 2D imaging configuration using 1D conformable arrays. However, in various embodiments the presented concepts can be readily extended to 3D imaging of the brain using conformable 2D arrays. This same approach could also be extended to imaging limbs (arms and legs), particularly for imaging lymphatic and vascular structures in limbs.

FIGS. 11A, 11B, 11C, and 11D are line drawings depicting the phased-emission stage of operation of an embodiment of PECT for 3D imaging in accordance with the present invention.

FIG. 11A depicts a 2D transmitting array 1102 comprising a square grid of ultrasonic transducers 1104, and a 2D receiving array 1106 comprising resistive electrode line (or strip) transducers 1108 for imaging the structure or properties of the 3D region existing between the two arrays. FIGS. 11B, 11C, and 11D depict various combinations and orientations of del beam projections (1112, 1120, 1128, 1132) to a single line transducer 1108 in the receiving array 1106.

FIG. 11B depicts an embodiment wherein the transmitting array elements 1104 are pulsed in groups 1114, each group 1110 consisting of 10 elements and aligned as a row in the y-direction of the square-grid array. In the depicted embodiment the 10 elements 1104 within each group 1110 are pulsed with different time-delays or phases to focus the ultrasonic emissions. As the pulses propagate through the tissue, they converge 1112 to a point 1116 onto a single line (or strip) detector element 1108 in the ultrasonic receiving array 1106, thus forming an inverted fan-beam, or del-beam, projection in the interrogated region or object.

In various embodiments resistive electrode line (or strip) receiver elements 1108 in the ultrasonic receiving array 1106 detect the voltage difference created at the focal point of the projections 1116. Although the intended x and y coordinates of the focal point 1116 may have been calculated in the beam forming step of the data acquisition process, unknown density and bulk modulus variations in the tissue could refract the focal point to different coordinates on the receiver array 1106. To account for refractive effects, since the receiver elements are discrete in the x-direction of the receiver array, the x-coordinate of the focal point may be determined by which line (or strip) element 1108 is activated. To determine the y-coordinate of the focal point, the line element may be coated with resistive electrodes. These electrodes may allow the y-position of the focal point 1116 to be ascertained by measuring the voltage differences from the electrodes at the two ends of the line element 1108.

FIG. 11C depicts projections 1120 generated by an embodiment of the transmitting array 1102 that are oriented in different directions than the projections 1112 in FIG. 11B. The projections 1120 of the depicted embodiment are produced by groups of transmitting elements aligned as a row in the x-direction 1118 of the square-grid array 1102. Although a plurality of projections 1122 all terminate at the same focal point 1124, they provide information from different slices of the interrogated region. FIG. 11D depicts the combining of projections as shown in FIG. 11B and FIG. 11C. Combining projections in this manner may provide greater information for imaging the structure and micropathology of the tissue at high resolution.

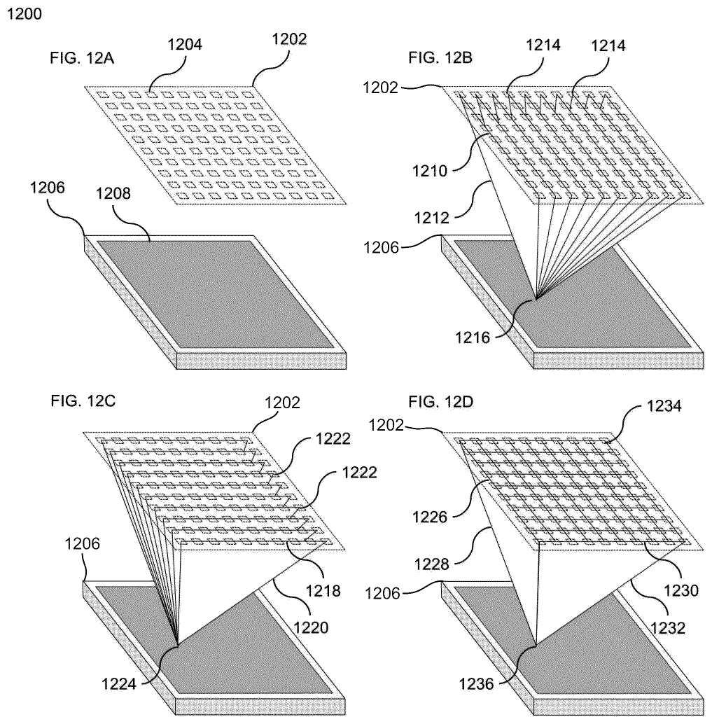

FIGS. 12A, 2B and 12C are line drawings depicting the phased-emission stage of operation of an embodiment of PECT for 3D imaging in accordance with the present invention.

FIG. 12A is a line drawing of a 2D transmitting array 1202, and a 2D receiver array 1206 with a single receiving element 1208 comprising a resistive electrode large-area (or plate) transducer for imaging the structure or properties of the 3D region existing between the two arrays 1202, 1206. FIG. 12B, FIG. 12C, and FIG. 12D depict embodiments of various combinations and orientations of del beam projections (1212, 1220, 1228, 1232) to a large-area transducer 1208 in the receiving array 1206.

In the embodiment depicted by FIG. 12B, the transmitting array elements 1204 are pulsed in groups 1214, each group 1210 consisting of 10 elements and aligned as a row in the y-direction of the square-grid array. The 10 elements 1204 within each group 1210 are pulsed with different time-delays or phases to focus the ultrasonic emissions. As the pulses propagate through the tissue, they converge 1212 to a point 1216 onto a large-area (or plate) detector element 1208 in the ultrasonic receiving array 1206, thus forming an inverted fan-beam, or del-beam, projection in the interrogated region or object.

In various embodiments the large-area (or plate) receiver element 1208 in the ultrasonic receiving array 1206 detects the voltage difference created at the focal point of the projections 1216. Although the intended x and y coordinates of the focal point 1216 have been calculated in the beam forming step of the data acquisition process, unknown density and bulk modulus variations in the tissue could refract the focal point to different coordinates on the receiver array 1206. To account for refractive effects and determine the x- and y-coordinates of the focal point, the plate element may be coated with resistive electrodes. These electrodes may allow the x- and y-positions of the focal point 1216 to be ascertained by measuring the voltage differences from the electrodes at the four corners of the plate element 1208.

FIG. 12C depicts an embodiment of projections 1220 generated by the transmitting array 1202 that are oriented in different directions than the projections 1212 in FIG. 12B. The projections 1220 are produced by groups of transmitting elements aligned as a row in the x-direction 1218 of the square-grid array 1202. Although in the depicted embodiment a plurality of projections 1222 all terminate at the same focal point 1224, they may provide information from different slices of the interrogated region. FIG. 12D depicts an embodiment of the combining of projections as shown in FIGS. 12B and 12C. Combining projections in this manner may provide greater information for imaging the structure and micropathology of the tissue at high resolution.

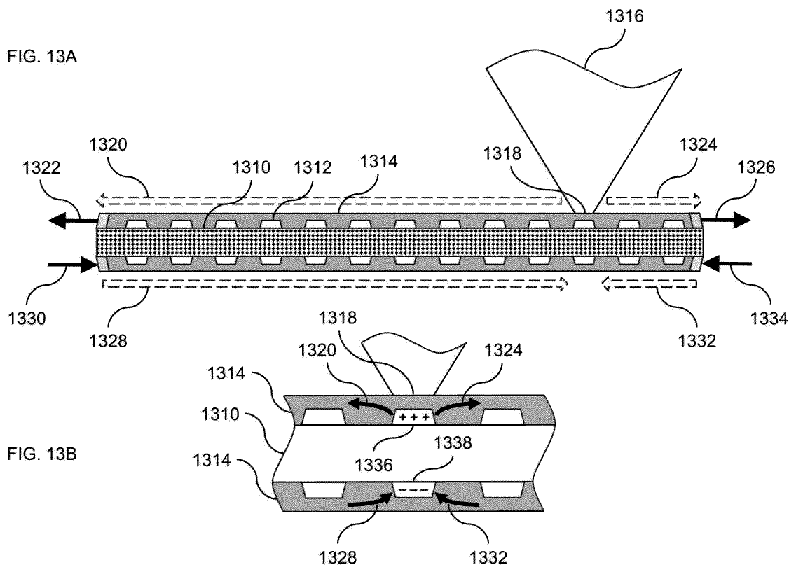

FIGS. 13A, and 13B depict a cross-section of an embodiment of a resistive electrode line (or strip) transducer in accordance with the present invention.

FIG. 13A shows the structure of the line transducer, including a piezoelectric material 1310; isolated, highly conductive electrode islands 1312 to collect charge from a localized, activated region on the piezoelectric material; and resistive electrodes 1314 to conduct the charge to the ends of the transducer element. FIG. 13B depicts a close-up of an embodiment of the mechanism of activating the line transducer.

In various embodiments the line transducer is activated when a del beam 1316 is focused 1318 on the line transducer. The focus point 1318 of the del beam 1316 activates a localized region of electrical charge separation on the piezoelectric material, which induces electrical charges to collect on the isolated, highly conductive islands 1336 and 1338. The charges flow from the isolated, highly conductive islands 1336 and 1338 to the ends of the line transducer through the resistive electrodes 1314. The dotted lines 1320, 1324, 1328, and 1332 show the direction of the electrical current flowing from the region of the piezoelectric transducer 1310 with the localized charge separation. In various embodiments as the charges flow through the resistive electrodes 1314, the voltage of the electrical current drops as a function of the resistivity of the resistive electrode 1314 material and the distance it travels down the electrode. The voltages may be read at the ends of the resistive electrodes 1322, 1326, 1330, and 1334 to determine the position of the del beam 1316 focus 1318 and resulting activated piezoelectric region.

The principles described above for the 1D resistive electrode line (or strip) transducer 1314 can be expanded to a 2D resistive electrode large-area (or plate) transducer. One embodiment of such a device would be a square 2D resistive electrode transducer, where the voltages are read at the corners of the square to determine the position of the del beam focus point.

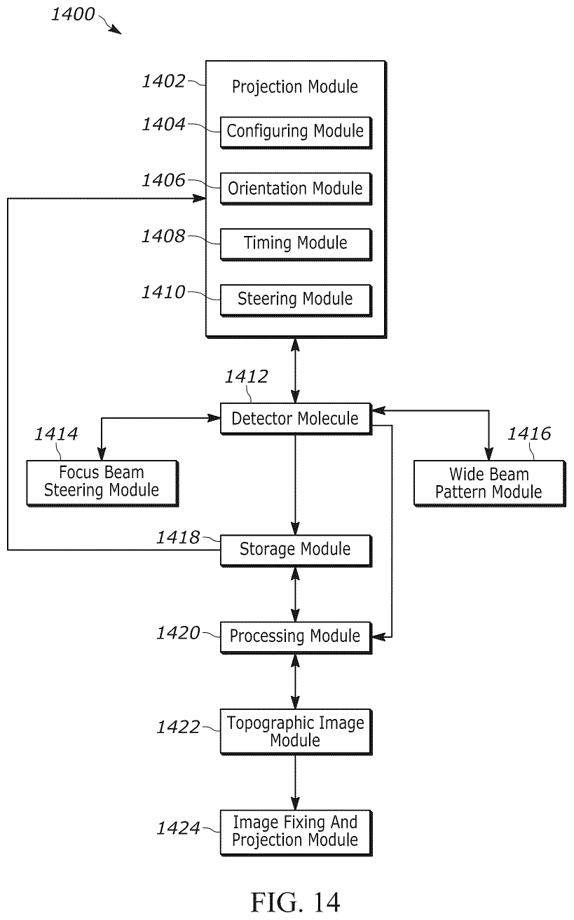

FIG. 14 is a software flow chart depicting an embodiment of a software program for an apparatus to interrogate the internal structure of a region or object in accordance with the present invention, wherein the software program comprises: a projection module 1402, a configuring module 1404, an orientation module 1406, a timing module 1408, a steering module 1410 a detector module 1412, a focus beam scanning module 1414, a wide beam pattern module 1416, a storage module 1418, a processing module 1420, and a tomographic image module 1422.

In some embodiments the configuring module 1404 configures electronic waves from the projection module 1402 as a triangular projection which orientation module 1406 inverts to form an invented fan or del beam. The timing module 1408 pulses a sequence of fan or del beams projection module 1402 configuring module 1404 configures an ultrasonic wave as a triangular projection 1402. The orientation module 1406 then may invert the triangular projection to create an inverted fan or del beam.

In certain embodiments the timing module 1408 pulses the projection of the del beams and the steering module 1410 electronically steers the inverted del beam focal point to a receiving array element in the detector module. 1412. The focus beam scanning module 414 may electronically scan the focus of the del beam to a plurality of elements in the receiving array, thus increasing the signal strength.

In certain embodiments the detector module 412 receives and reads a del beam focal point from a receiving array element. A focus beam scanning module may electronically scan the focus of the del beam to a plurality of elements in the receiving array. A storage module 1418 may collect and store signal information. In some embodiments a processing module 1420 processes the plurality of intersecting wide-beam patterns with a CT algorithm to reconstruct the ultrasonic transmission properties of the interrogated region or object. A tomographic image module may create a 3D image of the interrogated region or object. In certain embodiments an image fixing and projection module may record and/or distribute the image. In various embodiments any two or more modules may be merged or combined.

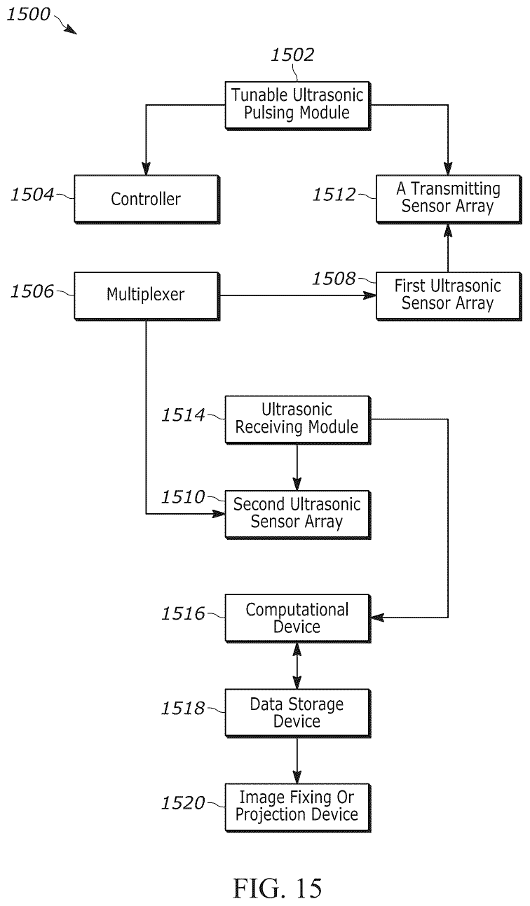

FIG. 15 is a diagram of an embodiment of a system to construct 2D and 3D ultrasonic images in attenuating media in accordance with the present invention, wherein the system comprises: a tunable ultrasonic pulsing module 1502, a controller 1504, a multiplexer 1506, a first electronic sensor array 1508, a second electronic sensor array 1510, a transmitting sensor array 1512, a multiplexer an ultrasonic receiving module 1514, a computational device 1516, a data storage device 1518, and an image fixing or projection device 1520.

In some embodiments the controller 1504 tunes the ultrasonic pulsing module 1502 for each frequency step in the multifrequency and/or broadband ultrasonic spectrum. In certain embodiments the ultrasonic pulsing module 1502 pulses the ultrasonic transmissions from the transmitting sensor array 1510. The multiplexer 1506 may switch through the sensors 1508, 1510 in the transmitting sensor array 1512 and select combinations of transmitting array elements to generate each ultrasonic beam geometry. In certain embodiments the first ultrasonic sensor array 1508 generates and broadcasts ultrasonic waves with multifrequency and/or broadband characteristics and the second ultrasonic sensor array receives and detects ultrasonic waves with multifrequency and/or broadband characteristics. The multiplexer 1506 may switch through the sensors in the receiving sensor array and select combinations of receiving array elements to correspond to each ultrasonic beam geometry.

In certain embodiments the ultrasonic receiving module 1514 to amplifies, filters, and/or rectifies the received ultrasonic signal from the selected receiving array elements. The computational device 1516 may that calculate the frequency steps for a multifrequency and/or broadband ultrasonic spectrum. Some embodiments comprise beam geometry combinations required to reconstruct a 2D or 3D image of the internal structure or property distribution of a medium and/or sensor element combinations for each ultrasonic beam geometry, and corresponding sensor element delays for each frequency step and for each beam geometry.

In certain embodiments the data storage device 1518 stores the frequency steps, beam geometries, sensor element combinations, and sensor element delays. The computational device 1520 may use the beam pattern geometries, receiving element measurements, and broadband ultrasonic spectrum analysis to construct a 2D or 3D image of the internal structure or property distribution of the medium. An image fixing and/or projection device 1520 sometimes captures and/or distributes the constructed image.

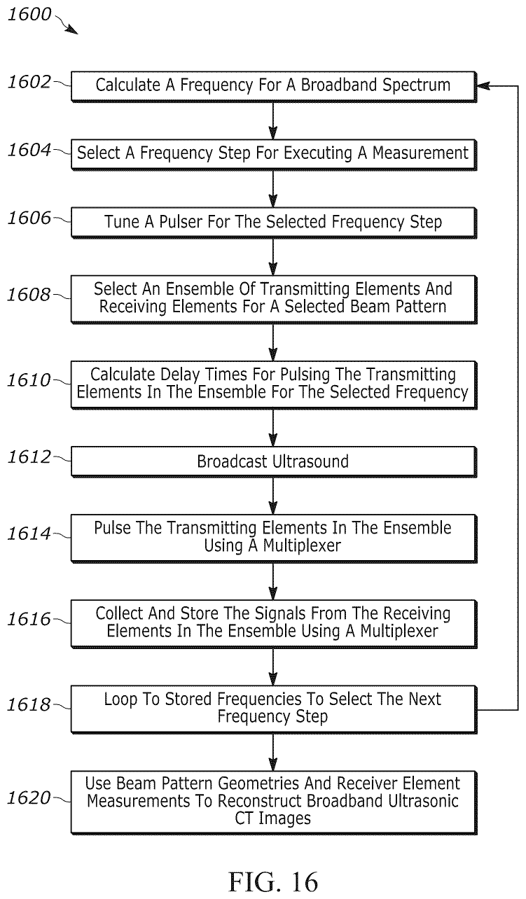

FIG. 16 is a flow chart depicting an embodiment of a method 1600 to increase the signal strength from a first transmitting sensor array by broadcasting ultrasound through an attenuating medium, to a second receiving sensor array, and obtaining two-dimensional and three-dimensional images of the internal structure or distribution of material, physical, chemical, or biological properties of the medium, in accordance with the present invention, the method comprising: calculate a frequency for a broadband spectrum 1602, select a frequency step for executing a measurement 1604, tune a pulser for the selected frequency step 1606, select an ensemble of transmitting elements and receiving elements for a selected beam pattern 1608; calculate delay times for pulsing the transmitting elements in the ensemble for the selected frequency 1610; broadcast ultrasound 1612, pulse the transmitting elements in the ensemble using a multiplexer 1614, collect and store the signals from the receiving elements in the ensemble using a multiplexer 1616, loop to stored frequencies to select the next frequency step 1618, use beam pattern geometries and receiver element measurements to reconstruct broadband ultrasonic CT images 1620.

In certain embodiments the broadband ultrasonic CT images are 2D, 3D or 4D. The attenuating medium may be any one or more of biological, mineral, geologic, rock, soil, landform, machine, construction, aqueous, or oceanic or other attenuating medium, including animal, human or other tissue. In some embodiments broadband spectrum analysis is used construct a 3D pathology image of a tested tissue or region. The method herein sometimes comprises the use of broadband spectrum analysis to construct a 3D inclusion image of the medium. An inclusion may comprise at least one of oil, precious metal, industrial metal, precious stones, water, or other inclusion. In various embodiments the method herein comprises the use of broadband spectrum analysis to interrogate a geological structure or feature wherein the geological structure or feature may comprise least one of a fault line, a sink hole, a cavern, a well, a rock type, a soil type, a geological stratum, an archaeological structure, or other geological structure or feature.

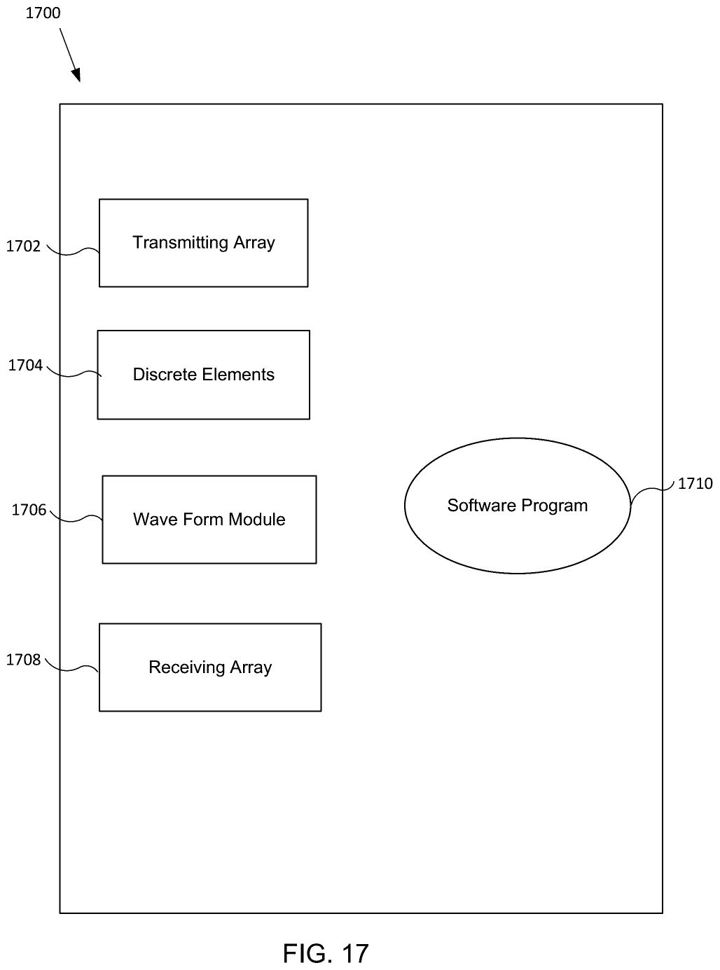

FIG. 17 is a line diagram depicting an apparatus to interrogate the internal structure of a region or object, the apparatus comprising a transmitting array 1702 of discrete elements 1704, a wave form module 1706, a receiving array 1708 and a software program 1710 to at least one of coordinate a phased pulsing of the transmitting elements, read the receiver elements, and topographically process the signals.

In certain embodiments the transmitting array 1702 transmits and focus electronic waves through an interrogated region or object. In some embodiments the electronic waves are ultrasonic. In various embodiments wave form module 1706 configures the ultrasonic waves as a triangular projection by phasing (timing) the emissions from each of the active discrete elements 1704. The triangular projection may comprise an inverted fan beam, or del beam (V). In various embodiments the software program 1710 at least one of coordinates a phased pulsing of the transmitting elements, reads the receiver elements, and topographically processes the signals.

The present invention may be embodied in other specific forms without departing from its spirit or essential characteristics. The described embodiments are to be considered in all respects only as illustrative and not restrictive. Elements of the technology provided herein may be merged or combined in different configurations and may interact with each other in various ways. Various embodiments may include different combinations of system elements and not all elements are required in every embodiment.

The scope of the invention is, therefore, indicated by the appended claims. All changes which come within the meaning and range of equivalency of the claims are to be embraced within their scope.

What is claimed is:

1. An apparatus to interrogate the internal structure of a region or object, the apparatus comprising:
a. a transmitting array of discrete elements to transmit and focus electronic waves through an interrogated region or object;
b. a wave form module to configure the ultrasonic waves as a triangular projection by phasing (timing) the emissions from each of the active discrete elements and to invert the triangular projection to create an inverted fan beam, or del beam (∇)
c. a receiving array for the electronic waves comprising at least one of a plurality of strip elements or a single large area element;
d. a software comprising a program to at least one of coordinate a phased pulsing of the transmitting elements, read the receiver elements, and topographically process the signals.

2. The apparatus of claim 1 wherein a 2D receiving array comprises resistive electrode line (or strip) transducers for imaging the structure or properties of the 3D region existing between the transmitting array and the receiving array.

3. The apparatus of claim 2 wherein a line element is coated with resistive electrodes to enable a y-position of a focal point to be ascertained by measuring the voltage differences from the electrodes at the two ends of the line element.

4. The apparatus of claim 1 wherein the electronic waves are ultrasonic.

5. The apparatus of claim 1 wherein the electronic waves comprise electromagnetic (EM) frequency bands with wavelengths amenable to phased array focusing, including radio waves (atmospheric and geologic imaging), microwaves (NDE of nonliving manufactured materials), or terahertz or far infrared (FIR) waves (micropathology imaging).

6. The apparatus of claim 5 further comprising combining HF ultrasonic elements with FIR elements in one or both of the transmitting and receiving arrays.

7. The apparatus of claim 1 wherein the software program comprises at least one of:
a. a projection module to broadcast a wave form;
b. a timing module to phase a plurality of wave transmissions;
c. a configuring module to configure the wave transmission as a triangular projection;
d. an orientation module to create an inverted del beam;
e. a receiving array module
f. a detector module to receive and read a del beam focal point from a receiving array element;
g. a steering module to electronically steer an inverted del beam focal point to a specific receiving array element;
h. a focus beam scanning module to electronically scan the del beam focal point to a plurality of receiving array elements;
i. a wide beam pattern module to scan phased beam patterns across the receiving array to produce a plurality of intersecting wide beam patterns;
j. a storage module to collect and store signals from the receiving array elements;
k. a processing module process the plurality of intersecting wide-beam patterns with a CT algorithm to reconstruct the ultrasonic transmission properties of the interrogated region or object; and
l. a tomographic image module to create a 3D image of the interrogated region or object.

8. The software program of claim 7 further comprising an image fixing or projection module to record and/or distribute the image.

9. A system to construct 2D and 3D ultrasonic images in attenuating media, where the ultrasonic measurements and data are multifrequency and/or broadband in nature, and the 2D/3D ultrasonic images retain the multifrequency and/or broadband characteristics of the ultrasonic measurements and/or data, and comprising:
a. a tunable ultrasonic pulsing module;
b. a controller that tunes the ultrasonic pulsing module for each selected frequency step in the multifrequency and/or broadband ultrasonic spectrum;
c. a multiplexer that switches through the sensors in a transmitting sensor array and selects combinations of transmitting array elements to generate each ultrasonic beam geometry;
d. a first ultrasonic sensor array that generates and broadcasts ultrasonic waves with multifrequency and/or broadband characteristics;
e. a second ultrasonic sensor array that receives and detects ultrasonic waves with multifrequency and/or broadband characteristics;
f. a multiplexer that switches through the sensors in the second ultrasonic sensor array and selects combinations of receiving array elements to correspond to each ultrasonic beam geometry;
g. an ultrasonic receiving module to amplify, filter, and/or rectify a received ultrasonic signal from the combinations of receiving array elements;
h. a computational device;
i. a data storage device that stores frequency steps, beam geometries, sensor element combinations, and sensor element delays; and
j. a computational device that uses the beam pattern geometries, receiving array element measurements, and broadband ultrasonic spectrum analysis to construct a 2D or 3D image of the internal structure or property distribution of the medium.

10. The system of claim 9 wherein the computational device calculates at least one of frequency steps for a multifrequency and/or broadband ultrasonic spectrum, beam geometry combinations required to reconstruct a 2D or 3D image of an internal structure or property distribution of a medium, sensor element combinations for each ultrasonic beam geometry, or corresponding sensor element delays for each frequency step or for each beam geometry.

11. The system of claim 10 further comprising an image fixing and/or projection device to capture and/or distribute a constructed image.

12. A method to increase the signal strength from a first transmitting sensor array, broadcasting a ultrasound through an attenuating medium, to a second receiving sensor array, and obtaining two-dimensional and three-dimensional images of the internal structure or distribution of material, physical, chemical, or biological properties of the medium, the method comprising:
a. calculate a frequency for a broadband spectrum;
b. select a frequency step for executing a measurement;
c. tune a pulser for the selected frequency step;
d. select an ensemble of transmitting elements and receiving elements for a selected beam pattern;
e. calculate delay times for pulsing the transmitting elements in the ensemble for the selected frequency;
f. broadcast ultrasound
g. pulse the transmitting elements in the ensemble using a multiplexer;
h. collect and store the signals from the receiving elements in the ensemble using a multiplexer;
i. loop to stored frequencies to select the next frequency step
j. use beam pattern geometries and receiver element measurements to reconstruct broadband CT images.

13. The method of claim 12 wherein a broadband ultrasonic CT image is 2D, 3D, or 4D.

14. The method of claim 13 wherein a use of both on-axis and off-axis phasing combinations focuses the ultrasonic pulses creating 2D projections are either perpendicular or non-perpendicular to a 2D, square-grid, transmitting array.

15. The method of claim 13 wherein the attenuating medium comprises animal tissue, human tissue, or other tissue.

16. The method of claim 15 further comprising use of broadband spectrum analysis to at least one of construct a 3D pathology image of a tested tissue or region and to ablate the region with the focused ultrasound intensity.

17. The method of claim 12 wherein the attenuating medium comprises at least one of biological, mineral, geologic, rock, soil, landform, machine, construction, metals, ceramics, glasses, polymers, elastomers, composites, manufactured objects and parts in nondestructive evaluation (NDE), or parcels and baggage in aviation security.

18. The method of claim 12 further comprising use of broadband spectrum analysis, including lower acoustic frequencies in the audible and infrasonic range, to construct a 3D image of an inclusion of the attenuating medium and wherein the inclusion comprises at least one of oil, metal, fossil, drill core, industrial metal, stones, water, or other inclusion.

19. The method of claim 11 further comprising oceanographic imaging with floating and submerged acoustic arrays, and geologic imaging of the Earth's upper crust including using cross-well borehole methods.

20. The method of claim 19 wherein the geological imaging is of least one of a fault line, a sink hole, a cavern, a well, a rock type, a soil type, a geological stratum, an archaeological structure or other geological structure or feature.

21. A method to construct 2D and 3D ultrasonic images in attenuating media, including a pathology, where the ultrasonic measurements and data are multifrequency and/or broadband in nature, and the 2D/3D ultrasonic images retain the multifrequency and/or broadband characteristics of the ultrasonic measurements and/or data, and comprising:
a. providing a first transmitting sensor array comprising multiple array elements arranged in a square or other configuration;
b. broadcasting ultrasound through an attenuating medium to a second receiving sensor array;
c. providing a first ultrasonic sensor array that generates and broadcasts ultrasonic waves with multifrequency and/or broadband characteristics;
d. providing a second ultrasonic sensor array that receives and detects ultrasonic waves with multifrequency and/or broadband characteristics, the second ultrasonic sensor array comprising at least one of a plurality of strip elements or a single large area element; and
e. obtaining two-dimensional and three-dimensional images of an internal structure or distribution of material, physical, chemical, biological, or other properties of the medium.
